(19) United States
(12) Patent Application Publication
(10) Pub. No.: US 2011/0190775 A1
(43) Pub. Date: Aug. 4, 2011

(54) Device and method for achieving accurate positioning of acetabular cup during total hip replacement

(76) Inventor: Keith J. Ure, Mount Shasta City, CA (US)

(21) Appl. No.: 12/698,443

(22) Filed: Feb. 2, 2010

(51) Int. Cl. A61B 17/56

(52) U.S. Cl. 606/91

(57) ABSTRACT

A method and device are provided in order to achieve desired orientation of an acetabular cup for total hip replacement or hip resurfacing. The method and device utilize preoperative CT, MRI or other scans, 3D computer modeling and a patient specific alignment jig created from CT, MRI or other data and computer 3D modeling. The device allows accurate placement of a drill hole to establish an acetabular axis and placement of an acetabular cup perpendicular to the axis.

### FIELD OF THE INVENTION

The present invention relates to joint implants, particularly hip joint implants.

### BACKGROUND OF THE INVENTION

Accurate acetabular cup position within the bony acetabulum is critical for successful function of a total hip prosthesis. Malposition of the cup can cause instability of the total hip implant, resulting in dislocation of the femoral head from within the cup. Malposition of the cup is also responsible for premature wear of the bearing surfaces. Both these events are a common cause for repeat surgery requiring revision of one or all of the components of a total hip replacement.

Ideal cup position is described by an inclination angle and an anteversion angle. Both are important in achieving ideal cup position. Prior art efforts for accurate acetabular cup positioning have included an alignment post on the device used to insert the acetabular cup. The alignment post typically is oriented at a 45 degree angle to the cup. Orientation of the alignment post vertically, in theory, would result in the cup being inserted into the acetabulum at a 45 degree inclination angle. However, multiple variables, such as patient position on the operating table, pelvic tilt, or poor estimation of whether the alignment post is truly vertical, can compromise accurate cup position.

Anteversion angle is typically achieved by a longitudinal rod attached to the cup insertion device. Anteversion is achieved by rotating the longitudinal rod about the transverse axis. Visual estimation of the cup position relative to the position of the acetabulum is also used as a guide. Such visual estimation is difficult due to multiple variables.

Computer assisted navigation has been utilized in an effort to achieve more accurate cup position. Such technology typically utilizes a computer, a tracking system to monitor position and points of the acetabulum and pelvis, and computer software which determines implant orientation relative to the tracking points of the acetabulum and pelvis. Its accuracy is limited due to multiple factors.

### OVERVIEW

A method and apparatus are provided for positioning an acetabular cup in a desired alignment in relation to a patient's hemipelvis. Using a patient-specific jig as a guide, bone is removed or displaced from the patient's hemipelvis to form a hole corresponding to a desired axis of alignment, and the acetabular cup is positioned in the desired alignment with the aid of a pin or post inserted into the hole. The hole may be located within the patient's acetabulum, and the pin or post may be affixed to the acetabular cup. To allow for positioning of the acetabular cup, the patient's acetabulum may be reamed, again with the aid of a pin or post inserted into the hole. The pin or post may be affixed to a reaming tool used for this purpose.

Alternatively, the pin or post may be located in a region outside the patient's acetabulum. The acetabular cup is impacted into the patient's acetabulum using an impactor rod having a laser attached thereto, and during impacting, the impactor rod may be manually guided so as to target the pin or post with a laser beam produced by the laser. The laser beam points along an axis that is parallel to and offset from an axis of force of the impactor rod. In other embodiments, both a primary (central) axis guide pin or post and a secondary (non-central) axis guide pin or post are used to achieved the desired alignment.

Additional features and benefits of the present invention will become apparent from the detailed description, figures and claims set forth below.

### BRIEF DESCRIPTION OF THE DRAWING

The present invention will be understood more fully from the detailed description given below, and from the accompanying drawings of various embodiments of the invention, which, however, should not be taken to limit the invention to the specific embodiments, but are for explanation and understanding only.

FIG. 1 is a perspective view of a hemipelvis and acetabulum, as well as a drill guide for establishing the axis of the acetabular implant.

FIG. 2 is a top perspective view of the acetabulum and drill guide of FIG. 1.

FIG. 3 is a perspective view of a hemipelvis and acetabulum showing placement of primary and secondary guide pins.

FIG. 4 is a perspective view of a hemipelvis and acetabulum showing cup placement using an impactor rod having a laser attached thereto.

### DETAILED DESCRIPTION

Those of ordinary skill in the art will realize that the following detailed description of the present invention is illustrative only, and is not intended to be in any way limiting. Other embodiments of the present invention will readily suggest themselves to such skilled persons having the benefit of this disclosure. It will be apparent to one skilled in the art that these specific details may not be required to practice present invention. In other instances, well-known devices are shown in block or schematic form to avoid obscuring the present invention. In the following description of the embodiments, substantially the same parts are denoted by the same reference numerals.

A device and method are provided whereby accurate cup position within the acetabulum, including both inclination and anteversion, may be achieved by means of computerized tomography scans (C.T. scans), magnetic resonance imaging (MRI), or other scans of the acetabulum obtained preoperatively, which may then be used to create an alignment jig placed within the acetabulum at the time of surgery.

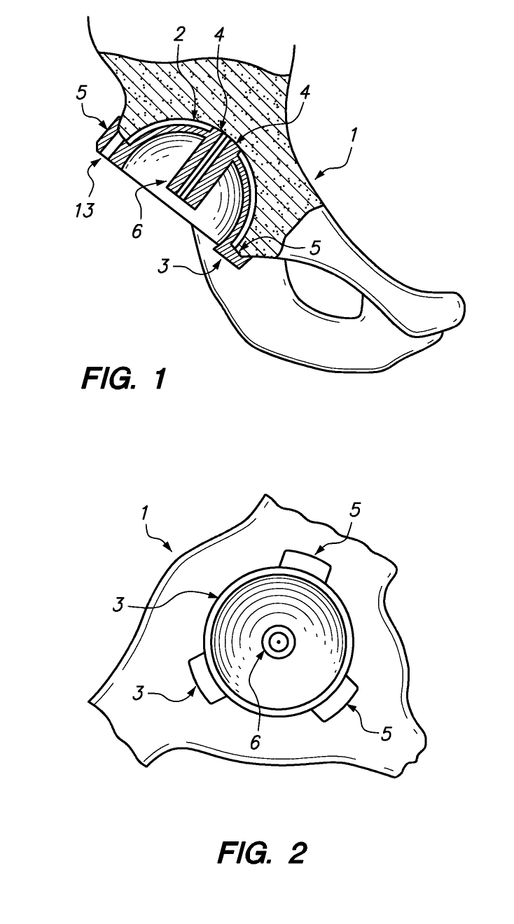

Referring now to FIG. 1 and FIG. 2, a perspective view is shown of a hemipelvis 1 and acetabulum 2, as well as a drill guide 3 for establishing a desired axis of the acetabular implant. A base 4 of a drill bit sleeve 6 and outer contact points 5 of the drill guide 3 may rest on the acetabulum or pelvis. A secondary drill bit sleeve 13 may also be provided.

The drill bit sleeve 6 of the drill guide orients a drill bit so as to achieve optimal orientation of a drill hole, thus establishing the desired acetabular axis for the acetabular implant. The orientation of the drill bit sleeve 6 of the drill guide will have been determined from preoperative CT, MRI or other scans of the patient's pelvis with respect to anteversion and inclination and allow determination of a desired acetabular axis in order to establish a desired position of the face of the acetabular cup relative to the acetabular axis.

The acetabular cup may be of a generally hemispherical design, and may have a protrusion, post or spike which may then be guided into the hole drilled into the acetabulum utilizing the drill guide 3.

The alignment jig is patient specific, and may be created prior to surgery based on preoperative CT, MRI or other scans and resulting data describing measurements and points of the patient's acetabulum and pelvis. The CT, MRI or other scans can accurately determine the diameter of the acetabulum and points on the acetabulum and pelvis for reference in order to create a patient specific jig. Data obtained from CT, MRI, etc. may be inputted into 3D computer assisted design software, after which a computer guided laser etcher or computer guided lathe creates the disposable jig by means of existing CAD technologies. Further details may be found in Roose, U.S. Patent Application 2005/0148843 entitled SYSTEM AND METHOD OF DESIGNING AND MANUFACTURING CUSTOMIZED INSTRUMENTATION FOR ACCURATE IMPLANTATION OF PROSTHESIS BY UTILIZING COMPUTED TOMOGRAPHY DATA, published Jul. 7, 2005, incorporated herein by reference.

The patient specific jig, placed within the acetabulum, has multiple contact points, for example contact points 5, on the margin and body of the jig, which rest on the acetabulum or pelvis so as to achieve a predetermined position on the acetabulum of the jig with respect to an X, Y and Z axis, and three planes in space relative to the axes. Such planes are typically referred to as sagittal, axial, and coronal planes.

The patient specific jig has a site for placing a drill hole within the dome of the patient's acetabulum. The orientation of the hole may be determined from the preoperative CT, MRI or other scans, which provide images of the acetabulum in axial, coronal and sagittal planes. Utilizing patient specific data from CT, MRI or other images, the orientation of the drill hole of the jig placed within the acetabulum may be made so as to create an axis of rotation for placement of the acetabular cup, as determined by the drill hole. Hence, the drill hole establishes the acetabular axis, and the acetabular axis serves to orient the implant cup. In one embodiment, the axis passes through the center of the implant socket and is perpendicular to the plane of the face of the acetabular cup.

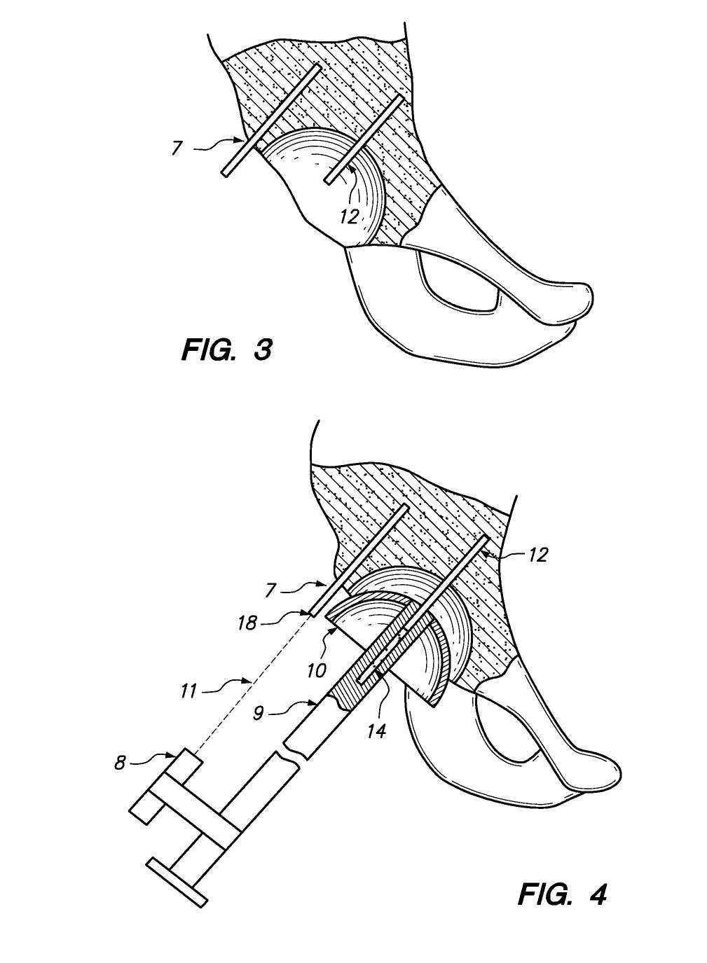

A central axis guide pin 12, which may be of the same diameter as the acetabular axis drill hole, may then be placed within the hole as shown in FIG. 3. The central axis guide pin 12 thus establishes the predetermined desired acetabular axis, established by preoperative CT, MRI or other scans and by the patient-matched jig 3 created from the scans.

A parallel pin guide, of a type commonly used in orthopedic surgery, may also be used to place a secondary axis guide pin 7, parallel to the central axis guide pin 12, into the bony pelvis. The secondary axis guide pin 12 may be placed into the pelvis just outside of the acetabulum, to serve as a second reference point in addition to the central axis guide pin 12.

Alternatively, the secondary axis guide pin 12 may be placed by means of a drill bit sleeve 13 in the patient specific jig 3, on a location of the jig outside of the acetabulum, oriented parallel to the drill bit sleeve 6.

The central axis guide pin 12, placed in the acetabulum by means of the patient-specific jig 3, may at this point be removed, or may serve as an axis post for reaming of the acetabulum, by means of acetabular reamers which have a cannulated center axis post, which passes over the axis pin. If the acetabular axis pin is removed, an acetabular reamer may itself be provided with a guide pin that may be inserted into the central axis hole.

Following acetabular reaming, the acetabular cup may be placed by any of various options.

Referring to FIG. 4, in one option the cup 10 may be created so as to have a central hole, the hole being perpendicular to the face of the cup. The central hole may be threaded, so as to accept an impaction rod 9, which may be threaded into the cup to be implanted. The impaction rod may be created so as to be cannulated with a cannula 14 having a diameter slightly larger than the central axis guide pin 12, thus allowing it to be passed over the central axis guide pin 12 in the desired acetabular axis, and perpendicular to the face of the cup. The acetabular cup may then be impacted into the prepared acetabular bone bed by means of the impaction post or rod 9.

Referring to FIG. 4, alternatively, or additionally, the cup 10 may be oriented by means of a laser pointer targeting device 8, attached to and parallel with the impaction rod 9 of the acetabular cup 10, which then targets the center of the secondary axis guide pin 7 placed outside of the acetabulum parallel to the central axis guide pin 12.

In accordance with this option, instead of referencing off the initial central axis guide pin 12, that pin is removed, and a secondary axis guide pin 7 is placed outside of the acetabulum and used as a reference. Removal of the central axis guide pin 12 initially placed within the acetabulum may at times be necessary due to inadequate bone to safely use the pin as a reference during cup impaction, or due to use of a solid metal cup which has no central axis hole option, such as a type used in metal-on-metal bearing total hip technology or metal on metal hip resurfacings.

Following removal of the central axis guide pin 12, the secondary axis guide pin 7, placed outside of the acetabulum, may be referenced by means of a compact laser target pointer 8, which may be attached to the acetabular cup impaction rod 9 and situated parallel to a central axis of the impaction rod 9 at a distance equal to a distance between the initial central axis guide pin 12 and the secondary axis guide pin 7. The laser pointer 8 may then be oriented so as to target the center of the flat end of the secondary axis guide pin 7. At such time as the impactor rod 9 is oriented so that the laser pointer 8 is centered on a head of the secondary axis guide pin 7, proper acetabular cup orientation has been achieved. The acetabular cup 10 may then be impacted into the acetabulum while maintaining the laser pointer 8 centered on the head of the secondary axis guide pin 7.

If, after having achieved satisfactory impaction and fixation of the acetabular cup, the laser pointer 8 is still centered on the center of the secondary axis guide pin 7, the proper acetabular cup axis will have been achieved, as determined by preoperative CT, MRI or other scans and by the 3D computer-modeled reference jig.

Another cup design that may be used with the present alignment techniques is described in U.S. patent application Ser. No. 12/429,167 of the present inventor titled A DEVICE AND METHOD FOR ACHIEVING ACCURATE POSITIONING OF ACETABULAR CUP DURING TOTAL HIP REPLACEMENT, filed Apr. 23, 2009, incorporated herein by reference. This cup is designed to take full advantage of the acetabular axis drill hole and achieve precise orientation of the acetabular cup relative to the sagittal, axial and coronal planes of the patient's pelvis.

Still another cup design that may be used with the present alignment techniques is described in U.S. patent application Ser. No. 11/772,698 titled PRECISION ACETABULAR MACHINING SYSTEM AND RESURFACING ACETABULAR IMPLANT, filed Jul. 2, 2007, incorporated herein by reference. The cup design may be modified such that in FIG. 7 thereof, the pin 62 instead of having a through-hole is solid, with no through-hole. Such a modification is believed to simplify proper joint lubrication.

While particular embodiments of the present invention have been shown and described, it will be obvious to those skilled in the art that, based on the teachings herein, changes and modifications may be made without departing from this invention and its broader aspects. Therefore, the appended claims are intended to encompass within their scope all such changes and modifications as are within the true spirit and scope of this invention.

What is claimed is:

1. A method of positioning an acetabular cup in a desired alignment in relation to a patient's hemipelvis, comprising:
using a patient-specific jig as a guide, removing or displacing bone from the patient's hemipelvis to form a hole corresponding to a desired axis of alignment; and
positioning the acetabular cup in the desired alignment with the aid of a pin or post inserted into the hole.

2. The method of claim 1, wherein the pin or post is affixed to the acetabular cup.

3. The method of claim 1, further comprising, to allow for positioning of the acetabular cup, reaming the patient's acetabulum with the aid of a pin or post inserted into the hole.

4. The method of claim 3, wherein the pin or post is affixed to a reaming tool.

5. The method of claim 1, wherein the pin or post is located in a region outside the patient's acetabulum.

6. The method of claim 5, further comprising:
impacting the acetabular cup into the patient's acetabulum using an impactor rod having a laser attached thereto; and
during said impacting, manually guiding the impactor rod so as to target the pin or post with a laser beam produced by the laser.

7. The method of claim 6, wherein the laser beam points along an axis that is parallel to and offset from an axis of force transmission of the impactor rod.

8. A method of placing an acetabular cup having a projecting member, comprising:
establishing an acetabular axis hole using a patient-specific jig; and
inserting the projecting member into the acetabular axis hole so as to achieve a desired orientation of a face of the acetabular cup.

9. The method of claim 8, wherein the projecting member is one of a pin, a post, a spike and a screw.

10. The method of claim 8, wherein the desired orientation is perpendicular to the projecting member.

11. A method of achieving correct orientation of an acetabular cup, comprising:
forming an acetabular axis drill hole within a patient's acetabulum and hemipelvis;
placing a central axis guide post within the acetabular axis drill hole;
placing an acetabular cup with the aid of the central axis guide post;
attaching to the acetabular cup an impactor rod; and
impacting the acetabular cup by advancing the impactor rod in line with a central axis of the central axis guide post.

12. The method of claim 11, further comprising:
placing a secondary axis guide post in bone surrounding the acetabulum, parallel to the central axis guide post; and
using the secondary axis guide post to guide placement of the acetabular cup.

13. A method of achieving correct orientation of an acetabular cup, comprising:
placing a central axis guide post within an area of a patient's acetabulum, thereby establishing a central axis;
placing a secondary axis guide post in bone surrounding the acetabulum, parallel to the central axis guide post, thereby establishing a secondary axis; and
using the secondary axis guide post to guide placement of the acetabular cup.

14. The method of claim 13, comprising:
attaching to the acetabular cup an impactor rod; and
impacting the acetabular cup by advancing the impactor rod in line with the central axis.

15. The method of claim 13, comprising:
providing a laser pointer attached to the impactor rod pointing parallel to an axis of the impactor rod;
wherein a distance between an axis of the laser pointer and the axis of the impactor rod is equal to a distance between the central axis and the secondary axis.

16. A method of achieving correct orientation of an acetabular cup, comprising:
establishing a central alignment axis;
placing an alignment target, thereby establishing a secondary alignment axis;
providing a laser pointer attached to an impactor rod pointing parallel to an axis of the impactor rod, wherein a distance between an axis of the laser pointer and the axis of the impactor rod is equal to a distance between the central alignment axis and the secondary alignment axis;
impacting the acetabular cup using the impactor rod while manually maintaining alignment of the laser pointer and the secondary alignment axis by viewing incidence of a laser beam upon the alignment target.

17. The method of claim 16, wherein the alignment target is a pin or post.

18. The method of claim 16, comprising:
placing a central axis guide post within an area of a patient's acetabulum, thereby establishing the central axis;
placing a secondary axis guide post in bone surrounding the acetabulum, parallel to the central axis guide post, thereby establishing the secondary axis.

19. The method of claim 16, comprising impacting the acetabular cup by advancing the impactor rod in line with the central axis.

20. An apparatus for impacting an acetabular cup, comprising:
an impactor rod; and
attached to the impactor rod, a laser, the laser producing a laser beam, the laser beam pointing along an axis that is parallel to and offset from an axis of force transmission of the impactor rod.

21. A method of acetabular reaming using a cannulated reamer having a cannula that fits over a pin or post, comprising;
establishing with respect to a patient's acetabulum a desired acetabular axis;
inserting into the patient's acetabulum a pin or post aligned with the desired acetabular axis;
fitting the cannula of the cannulated reamer over the pin or post; and
reaming the patient's acetabulum using the pin or post as a centering guide.

22. A cannulated reamer for acetabular reaming, comprising:
a hemispherical reaming member having a reaming surface on an outside diameter thereof; and
coupled to the hemispherical reaming member, a cannulated axial member having a cannula that fits over a pin or post to provide a centering action during reaming of a patient's acetabulum.
